(19) United States
(12) Patent Application Publication
(10) Pub. No.: US 2025/0190410 A1
(43) Pub. Date: Jun. 12, 2025

(54) MEASUREMENT DEVICE, CONTROL METHOD, AND CONTROL RECORDING MEDIUM

(71) Applicant: OMRON HEALTHCARE Co., Ltd., Kyoto (JP)

(72) Inventors: Kazuki KUMAGAI, Kyoto (JP); Yasunori FUKUDA, Kyoto (JP); Nozomi NAKAWAKI, Kyoto (JP); Yuya HIGUCHI, Kyoto (JP)

(21) Appl. No.: 19/058,936

(22) Filed: Feb. 20, 2025

Related U.S. Application Data
(63) Continuation of application No. PCT/JP2023/036916, filed on Oct. 11, 2023.

(30) Foreign Application Priority Data
Jan. 27, 2023 (JP) 2023-011008

(51) Int. Cl. G06F 16/215; G06F 16/23; G16H 40/67

(52) U.S. Cl. CPC G06F 16/215; G06F 16/2365; G16H 40/67

(57) ABSTRACT

A measurement device, a control method, and a control program that can improve analysis efficiency of biological data are provided. A measurement device according to one aspect of the present invention includes a control unit (30) that performs measurement based on biological data obtained by a sensor and wireless communication with an information terminal (5). Then, the control unit (30) assigns an error detection code in a variable data length unit corresponding to content of the biological data to the biological data obtained by sensing by the sensor, and transmits, to the information terminal (5), the biological data with the error detection code assigned.

### CROSS-REFERENCE TO RELATED APPLICATIONS

This application is the U.S. national stage application filed pursuant to 35 U.S.C. 365 (c) and 120 as a continuation of International Patent Application No. PCT/JP2023/036916, filed Oct. 11, 2023, which application claims priority to Japanese Patent Application No. 2023-011008, filed Jan. 27, 2023, which applications are incorporated herein by reference in their entireties.

### TECHNICAL FIELD

The present invention relates to a measurement device, a control method, and a control program.

### BACKGROUND ART

There is a known bioinformation processor including: a control unit that outputs detection values for a predetermined time of bioinformation and an error detection code corresponding to the detection values for the predetermined time; and an arithmetic unit that outputs, to the control unit, secondary information (pulse rate) related to a biological body obtained based on the detection values for the predetermined time output by the control unit and verification information indicating presence/absence of an error detected based on the error detection code (Patent Document 1).

### CITATION LIST

### Patent Literature

Patent Document 1: JP 2009-195447 A

### SUMMARY

### Technical Problem

In a case where pulse wave data measured by a health device is saved in a storage memory, if noise occurs in a communication path or the pulse wave data is garbled after saved in the storage memory, it is not possible to determine as to which part of the pulse wave data to be erroneous when the pulse wave data is extracted and analyzed thereafter. Therefore, it is difficult to remove erroneous data from among measurement data, and there is a case where accurate analysis of pulse wave data cannot be performed.

In a configuration where error detection is performed in a predetermined time unit, in a case where one piece of data in the predetermined time unit has a plurality of pieces of bioinformation (e.g., information of a plurality of pulses), when an error is detected in the data, even though one piece of bioinformation is erroneous, other valid biological data are also discarded, and the analysis efficiency of the biological data may be deteriorated.

According to the bioinformation processor of Patent Document 1, it is possible to verify whether or not bioinformation having an error is included somewhere in bioinformation for a predetermined time. However, detailed error determination other than a predetermined time cycle is not described.

The present invention has been made in view of such circumstances in one aspect, and an object thereof is to provide a measurement device, a control method, and a control program that can improve analysis efficiency of biological data.

### Solution to Problem

The present invention employs the following configurations to solve the above-described problems.

(1)

A measurement device including:
a control unit that performs measurement based on biological data obtained by a sensor and wireless communication with an information terminal,
in which the control unit
assigns an error detection code in a variable data length unit corresponding to content of the biological data to the biological data obtained by sensing by the sensor, and
transmits, to the information terminal, the biological data with the error detection code assigned.

According to (1), for example, in analysis of biological data in an information terminal such as a smartphone or a cloud server, even if there is an error in the biological data, the error can be detected in a meaningful data unit corresponding to the content of the biological data, and thus the efficiency of the analysis can be improved.

(2)

The measurement device according to (1), in which
the variable data length unit is different from a transmission unit of the biological data to the information terminal.

As in (2), by assigning an error detection code in a data length unit different from a transmission unit of biological data to the information terminal, it is possible to improve the analysis efficiency of the biological data.

(3)

The measurement device according to (1) or (2), in which
the biological data has cyclicity, and
the variable data length unit is a unit corresponding to a cycle of the biological data.

As in (3), in order to improve the analysis efficiency of the biological data, for example, it is preferable to assign an error detection code in units corresponding to the cycle of the biological data.

(4)

The measurement device according to any of (1) to (3), in which
the biological data includes sensing data obtained by the sensor and information of time when the sensing data is obtained, and
the variable data length unit is a unit based on a boundary between the sensing data and the information of the time.

As in (4), in order to improve the analysis efficiency of the biological data, for example, it is preferable to assign an error detection code in units based on a boundary between sensing data and time information.

(5)

The measurement device according to any of (1) to (4), in which
the variable data length unit is a unit based on a stage of the measurement.

As in (5), in order to improve the analysis efficiency of the biological data, for example, it is preferable to assign an error detection code in units based on a measurement stage of the biological data obtained by a sensor.

(6)

The measurement device according to any of (1) to (5), in which
the control unit includes
a first processor that performs the measurement,
a second processor that performs wireless communication with the information terminal, and
a nonvolatile memory connected to the second processor,
the first processor
sequentially transmits, to the second processor, the biological data obtained during sensing by the sensor and writes the biological data into the nonvolatile memory, and
the second processor
transmits, to the information terminal, the biological data written in the nonvolatile memory.

According to (6), by providing the second processor that performs wireless communication separately from the first processor that performs measurement based on the biological data, it is possible to distribute, to the second processor, the processing load for transmitting, to the information terminal, biological data such as pulse wave data, and reduce the processing load of the first processor. This can suppress a delay in processing such as measurement by the first processor. Note that the pulse wave may be a pressure pulse wave obtained by measuring a change in pressure applied to a blood vessel, or may be a volume pulse wave obtained by measuring a change in blood volume in the blood vessel.

(7)

The measurement device according to (6), in which
the variable data length unit is different from a transfer unit of the biological data from the first processor to the second processor.

As in (7), by assigning an error detection code in a data length unit different from a transfer unit of biological data from the first processor to the second processor, it is possible to improve the analysis efficiency of the biological data.

(8)

The measurement device according to (6) or (7), in which the first processor assigns the error detection code to the biological data.

As in (8), it is preferable for the first processor to assign an error detection code to biological data in a variable length.

(9)

The measurement device according to (6) or (7), in which
the first processor transmits information indicating the variable data length unit to the second processor based on content of the biological data, and the second processor assigns the error detection code to the biological data based on information indicating the variable data length unit.

According to (9), since the second processor performs calculation of an error detection code as compared with a case where the first processor performs determination of the biological data and calculation of the error detection code, it is possible to reduce the load on the first processor.

(10)

The measurement device according to any of (6) to (9), in which
the second processor assigns the error detection code to the biological data to be written into the nonvolatile memory.

As in (10), the timing at which the second processor assigns an error detection code to the biological data is preferably, for example, before writing the biological data into the nonvolatile memory.

(11)

The measurement device according to any of (6) to (9), in which
the second processor assigns the error detection code to the biological data read from the nonvolatile memory and transmitted to the information terminal.

According to (11), since data transmitted from the first processor to the second processor is written as it is into the nonvolatile memory by assigning an error detection code when the biological data is read from the nonvolatile memory, the first processor can perform address specification in the nonvolatile memory in no consideration of the data size that changes due to the assignment of the error detection code.

(12)

The measurement device according to any of (6) to (11), in which
the first processor
specifies an address of a write destination in the nonvolatile memory and causes the second processor to write the biological data into the nonvolatile memory, and specifies an address of a read source in the nonvolatile memory and causes the second processor to read the biological data from the nonvolatile memory and transmit the biological data to the information terminal.

According to (12), since the first processor is configured to specify the address of the nonvolatile memory connected to the second processor and instruct the second processor to write, read, and transmit the biological data, flow control and delivery confirmation are unnecessary in an interface (universal asynchronous receiver transmitter (UART) or the like) between the first processor and the second processor, and the transfer speed of the biological data to the information terminal can be improved. Since the second processor may perform writing of information to a specified address in the nonvolatile memory and reading and transmission of information from the specified address in the nonvolatile memory, the second processor can have a simple configuration. By an instruction to the second processor, the first processor can flexibly perform writing of the biological data to the nonvolatile memory, reading of the biological data from the nonvolatile memory, and transmission of the read biological data. However, since it is not necessary for the first processor itself to perform processing with a large load such as write processing, read processing, and transmission processing of biological data and the like, it is possible to reduce the processing load of the first processor as in (6) described above.

(13)

The measurement device according to (12), in which
the nonvolatile memory has an area allocated for the biological data, and the address of the write destination and the address of the read source are addresses in the area.

According to (13), by providing the nonvolatile memory with an area where information other than the biological data is not written, it is possible to suppress interference between writing of the biological data by an instruction from the first processor to the second processor and writing of other information by the second processor.

(14)

The measurement device according to any of (6) to (13), in which
the nonvolatile memory is inaccessible from the first processor.

According to (14), as compared with a configuration in which the first processor and the second processor share one nonvolatile memory, access processing can be distributed and sped up.

(15)

The measurement device according to any of (1) to (14), in which
the biological data is pulse wave data.

As in (15), as the biological data to be measured by the measurement device, for example, pulse wave data such as a pressure pulse wave and a volume pulse wave is preferable.

(16)

The measurement device according to (15), in which
the control unit outputs a blood pressure measurement result based on the pulse wave data.

According to (16), the first processor can perform wireless transmission of the pulse wave data to the information terminal and output of the blood pressure measurement result.

(17)

A control method of a measurement device including a control unit that performs measurement based on biological data obtained by a sensor and wireless communication with an information terminal,
in which the control unit
assigns an error detection code in a variable data length unit corresponding to content of the biological data to the biological data obtained by sensing by the sensor, and
transmits, to the information terminal, the biological data with the error detection code assigned.

According to (17), for example, in analysis of biological data in an information terminal such as a smartphone or a cloud server, even if there is an error in the biological data, the error can be detected in a meaningful data unit corresponding to the content of the biological data, and thus the efficiency of the analysis can be improved.

(18)

A control program of a measurement device including a control unit that performs measurement based on biological data obtained by a sensor and wireless communication with an information terminal, the control program for causing
the control unit to execute processing of
assigning an error detection code in a variable data length unit corresponding to content of the biological data to the biological data obtained by sensing by the sensor, and
transmitting, to the information terminal, the biological data with the error detection code assigned.

According to (18), for example, in analysis of biological data in an information terminal such as a smartphone or a cloud server, even if there is an error in the biological data, the error can be detected in a meaningful data unit corresponding to the content of the biological data, and thus the efficiency of the analysis can be improved.

### Advantageous Effects of Invention

According to the present invention, it is possible to provide a measurement device, a control method, and a control program that can improve analysis efficiency of biological data.

### BRIEF DESCRIPTION OF DRAWINGS

FIG. 1 is a view illustrating an information management system including a measurement device of the present invention and an information terminal that performs wireless communication with the measurement device.

FIG. 2 is a view illustrating a sphygmomanometer that is an example of the measurement device.

FIG. 3 is a view illustrating an example in which the information terminal is connected to a network.

FIG. 4 is a block diagram illustrating a configuration of the measurement device.

FIG. 5 is a block diagram illustrating a configuration of the information terminal.

FIG. 6 is a sequence diagram illustrating operations of a main MCU, a communication IC, and a nonvolatile memory in the measurement device.

FIG. 7 is a view illustrating an example of biological data to be measured by the measurement device.

FIG. 8 is a view illustrating an example in which the main MCU assigns an error detection code to the biological data.

FIG. 9 is a view illustrating an example in which the communication IC assigns an error detection code to the biological data.

### DESCRIPTION OF EMBODIMENTS

An embodiment according to one aspect of the present invention will be described below based on the drawings.

### § 1 Application Example

<Information Management System 100 Applied with Present Invention>

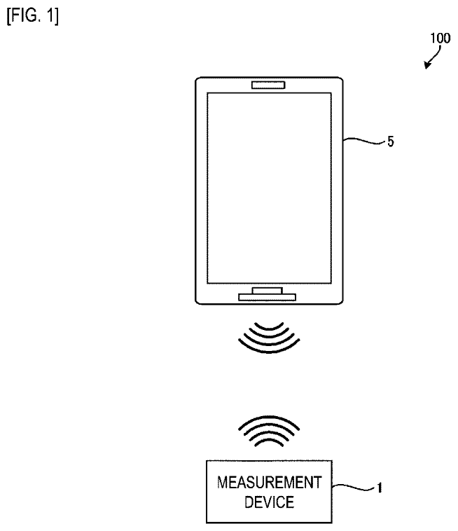

FIG. 1 illustrates an information management system 100 including a measurement device 1 of the present invention and an information terminal 5 that performs wireless communication with the measurement device 1.

The measurement device 1 includes a biological data measurement device that measures biological data such as a body weight, a body composition, blood pressure, a pulse, a heart rate, body temperature, blood glucose, or a blood oxygen saturation level. The measurement device 1 includes a measurement sensor for measuring a measurement target amount. The measurement target amount of the measurement sensor includes biological data such as a body weight, a body fat percentage, a blood pressure value, a pulse rate, a heart rate, body temperature, a blood glucose level, or a blood oxygen saturation level in accordance with the measurement device 1. The measurement device 1 is a non-wearable measurement device. The non-wearable measurement device is a measurement device that is not wearable. The wearable measurement device is a measurement device (e.g., an activity meter) carried by being worn on the body of a user. For example, the measurement device 1 (non-wearable measurement device) is a measurement device such as a scale, a body composition meter, a body weight-composition meter, or a sphygmomanometer that is used in a state of being installed on a ground or a table. The measurement device 1 transmits the measured biological data as measurement biological data of the user to the information terminal 5 by wireless communication.

The information terminal 5 stores the measurement biological data received from the measurement device 1 into a data storage unit in the information terminal 5. The information terminal 5 can perform wireless communication also with an external device other than the measurement device 1, and stores information acquired from the external device into the data storage unit in the information terminal 5. The information terminal 5 is an information processing device that analyzes various types of information acquired from the measurement device 1 and other external devices. The information terminal 5 is a terminal having a display such as, for example, a smartphone, a tablet terminal, a laptop computer, a desktop computer, or a wearable terminal. The information terminal 5 may be set so as to acquire measurement biological data from a specific measurement device 1. The specific measurement device 1 from which the measurement biological data is acquired may be registered in advance in the data storage unit of the information terminal 5.

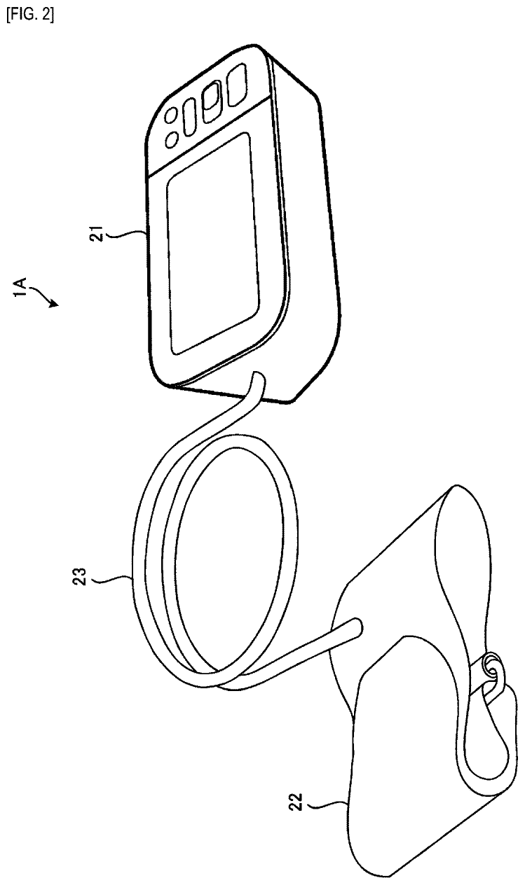

FIG. 2 is a view illustrating a sphygmomanometer 1A that is an example of the measurement device 1. The sphygmomanometer 1A is an example of a biological data measurement device, measures a blood pressure (pressure pulse wave data) of the user, and outputs a measurement result thereof to the user. The sphygmomanometer 1A transmits the measurement result as measurement biological data of the user to the information terminal 5 by wireless communication. For example, the sphygmomanometer 1A includes a main body portion 21, a cuff 22 that can be wound around an upper arm of the user, and an air tube 23 that connects the main body portion 21 and the cuff 22. In the example of FIG. 2, the cuff 22 and the main body portion 21 are separated, but the cuff 22 may be integrated with the main body portion 21.

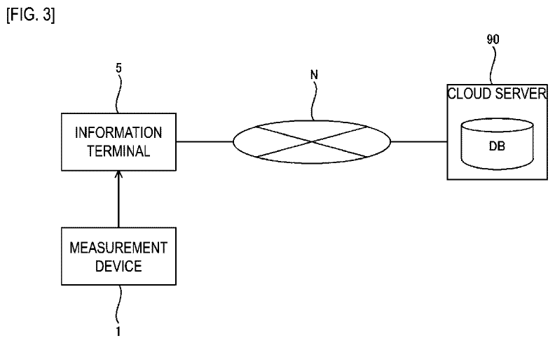

FIG. 3 is a view illustrating an example in which the information terminal 5 is connected to the network. As illustrated in FIG. 3, the information terminal 5 may be connected to a cloud server 90 via a wide area network N such as the Internet. The information terminal 5 may transmit the measurement biological data stored in the information terminal 5 to the cloud server 90 via the wide area network N, and the cloud server 90 may manage the measurement biological data of the user as a database. The information terminal 5 may acquire, via the wide area network N, the measurement biological data managed by the cloud server 90 and use the acquired measurement biological data.

### § 2 Configuration Example

### <Configuration of Measurement Device 1>

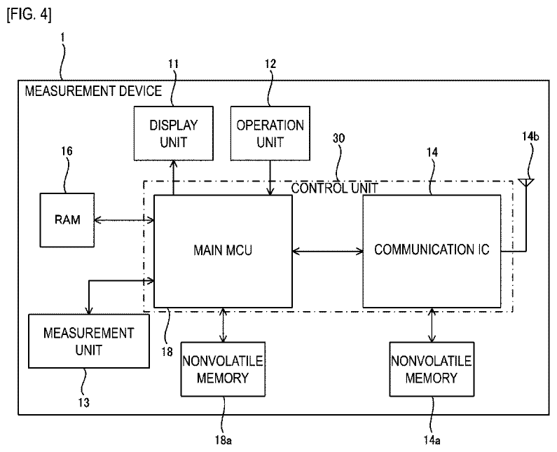

FIG. 4 is a block diagram illustrating the configuration of the measurement device 1. The measurement device 1 includes a display unit 11 that can display various types of information, an operation unit 12 that can be operated by the user, a measurement unit 13 that measures biological data and the like, a communication integrated circuit (IC) 14 that performs communication with an external device, a nonvolatile memory 14a connected to the communication IC 14, and a communication antenna 14b. The measurement device 1 includes a random access memory (RAM) 16 that temporarily stores information, a main micro controller unit (MCU) 18 that controls the operation of the entire device, and a nonvolatile memory 18a connected to the main MCU 18. The main MCU 18 is an example of the first processor of the present invention. The communication IC 14 is an example of the second processor of the present invention. A combination of the main MCU 18 and the communication IC 14 is an example of a control unit 30 of the present invention. As a communication interface between the main MCU 18 and the communication IC 14, for example, an interface such as a UART is used.

The display unit 11 includes a liquid crystal display or an organic electro luminescence (EL) display, for example. The operation unit 12 is a user interface that receives a user operation such as a button or a touch panel. The button includes a button physically provided at the measurement device 1 or a virtual button displayed on the display unit 11.

The measurement unit 13 includes a sensor that measures biological data such as a body weight, a body composition, blood pressure, a pulse, a heart rate, body temperature, blood glucose, and a blood oxygen saturation level. What to be measured varies depending on a measurement target of the measurement device 1.

The nonvolatile memory 14a is a recording medium that stores a parameter necessary for implementing a predetermined function, a control program, and biological data measured by the measurement unit 13. The nonvolatile memory 14a includes, for example, a flash memory. The nonvolatile memory 14a is provided with a biological data area allocated for storing biological data. The biological data stored in the nonvolatile memory 14a is managed by the communication IC 14.

The communication IC 14 implements a predetermined function by executing a control program. For example, the communication IC 14 can perform near-field wireless communication by executing a communication program stored in the nonvolatile memory 14a. The communication IC 14 performs communication in compliance with, for example, Bluetooth Low Energy (BLE (registered trademark)) standard. The communication IC 14 transmits, in a cyclic period, an advertise signal for performing wireless communication to an unspecified number of external devices by broadcast communication. The communication IC 14 sends an advertise signal including, for example, the name and attribute information of the measurement device 1. The BLE communication performed by the communication IC 14 is, for example, communication using a 2.4 GHz frequency.

The communication IC 14 can manage the biological data by executing, for example, a management program stored in the nonvolatile memory 14a. The biological data is biological data of the user measured by the measurement unit 13.

For example, the communication IC 14 performs write processing of writing the measured biological data into the nonvolatile memory 14a. The communication IC 14 performs read processing of reading the biological data from the nonvolatile memory 14a. The communication IC 14 writes the biological data into the nonvolatile memory 14a in accordance with a write instruction signal transmitted from the main MCU 18 to the communication IC, and reads the biological data from the nonvolatile memory 14a in accordance with a read instruction signal. The communication IC 14 performs write processing and read processing of the biological data with respect to the biological data area of the nonvolatile memory 14a. In the biological data area, information other than the biological data to be written in accordance with the write instruction signal from the main MCU 18 is not written. The biological data area is a dedicated area that can be used by the main MCU 18 in the area provided in the nonvolatile memory 14a.

The communication IC 14 performs transmission processing of transmitting the biological data read from the nonvolatile memory 14a to, for example, the information terminal 5 by wireless communication using the antenna 14b. The communication IC 14 performs transmission processing of the biological data in accordance with the transmission instruction signal transmitted from the main MCU 18 to the communication IC 14.

The RAM 16 includes a semiconductor device such as, for example, a dynamic RAM (DRAM) or a static RAM (SRAM), temporarily stores information, and also operates as a work area of the main MCU 18.

The nonvolatile memory 18a is a recording medium that stores a parameter necessary for implementing a predetermined function, a control program, address information of a biological data area in the nonvolatile memory 14a connected to the communication IC 14, and the like. The nonvolatile memory 18a includes, for example, an electrically erasable programmable read only memory (EEPROM). Note that in the present example, the nonvolatile memory 14a has a configuration independent of the communication IC 14, but for example, the nonvolatile memory 14a may be one module with the communication IC 14.

The main MCU 18 implements a predetermined function by executing a control program. For example, the main MCU 18 can perform measurement based on the biological data acquired by the measurement unit 13 by executing a measurement program stored in the nonvolatile memory 18a.

The main MCU 18 can manage and instruct the measured biological data by executing, for example, a management instruction program stored in the nonvolatile memory 18a. For example, the main MCU 18 transmits, to the communication IC 14, a write instruction signal for specifying an address of a write destination in the biological data area of the nonvolatile memory 14a and writing the biological data into the nonvolatile memory 14a. The main MCU 18 sequentially transmits the biological data obtained during the measurement by the measurement unit 13 to the communication IC 14 together with the write instruction signal. The main MCU 18 sequentially transmits the biological data to the communication IC 14 without receiving a response signal from the communication IC 14 to transmission of the biological data to the communication IC 14, that is, without performing delivery confirmation of the biological data. The sequential transmission of the biological data is to divide, at regular time intervals, and sequentially transmit the biological data, which is time-series data. When sequentially sending the biological data divided at regular time intervals, the main MCU 18 performs transmission (e.g., transmission by a streaming method) without performing delivery confirmation each time. The main MCU 18 attaches flag information indicating that the delivery confirmation as to whether or not the biological data has been delivered is not performed to the biological data and transmits the biological data to the communication IC 14. For example, the main MCU 18 transmits the biological data to the communication IC in 18 bytes every 32 msec.

The main MCU 18 transmits, to the communication IC 14, a read instruction signal for specifying an address of a read source in the biological data area of the nonvolatile memory 14a and reading the biological data from the nonvolatile memory 14a. Note that the address specification of write and read may be, for example, specification of a start address of write and read and specification of a size of write information or read information in the biological data area of the nonvolatile memory 14a, or specification of a start address and an end address of write and read.

The main MCU 18 can instruct the transmission processing of the communication IC 14 by executing, for example, a transmission instruction program stored in the nonvolatile memory 18a. For example, the main MCU 18 transmits, to the communication IC 14, a transmission instruction signal for transmitting an advertise signal for wireless communication (BLE communication) in a cyclic period, and a transmission instruction signal for transmitting biological data read from the nonvolatile memory 14a to an external device such as the information terminal 5. When writing the measured biological data into the nonvolatile memory 14a, when reading the biological data, and when transmitting the biological data to the external device, the main MCU 18 only transmits, to the communication IC 14, an instruction signal including address specification of the nonvolatile memory 14a. Then, the write processing of the biological data to the nonvolatile memory 14a, the read processing of the biological data, and the transmission processing of the biological data to the external device are configured to be executed by the communication IC 14 having received an instruction from the main MCU 18. That is, the main MCU 18 is configured to be indirectly accessible to the nonvolatile memory 14a via the communication IC 14, but not directly accessible to the nonvolatile memory.

Before sensing of the biological data, the main MCU 18 transmits, to the communication IC 14, a saving start instruction signal that instructs starting of saving of the biological data into the nonvolatile memory 14a. After sensing of the biological data, the main MCU 18 transmits, to the communication IC 14, a saving end instruction signal that instructs ending of the saving of the biological data into the nonvolatile memory 14a. After the sensing of the biological data ends, the main MCU 18 receives, from the communication IC 14, result information regarding writing of the biological data into the nonvolatile memory 14a. The result information is transmitted by being included in a response signal from the communication IC 14 to the main MCU 18 in response to the instruction of ending the saving. The result information includes information indicating the number of receptions of the biological data that the communication IC 14 has received from the main MCU 18 and information indicating the number of failures of the biological data that the communication IC 14 has failed to write into the nonvolatile memory 14a. Note that the result information may be information indicating the number of successes of the biological data successfully written into the nonvolatile memory 14a.

The main MCU 18 assigns an error detection code to the biological data obtained by sensing (pressurization and measurement) by the measurement unit 13. The error detection code is a code assigned so that an external device that receives the biological data can detect a data transmission error. The main MCU 18 calculates the error detection code by calculating the biological data in a certain procedure. The error detection code to be assigned is not particularly limited as long as the data transmission error can be detected. For example, the error detection code may be a cyclic redundancy check (CRC) code, a parity code, a checksum, or the like. The data transmission error includes, for example, an error in communication from the main MCU 18 to the communication IC 14, and an error at the time of writing, saving, or reading from the main MCU 18 to the nonvolatile memory 14a of the communication IC 14.

The main MCU 18 assigns an error detection code in a variable data length unit corresponding to the content of the biological data obtained by sensing. For example, the variable data length unit is a unit corresponding to the cycle of the biological data to be measured. Specifically, when the biological data obtained by sensing (pressurization and measurement) is pressure pulse wave data, the variable data length unit may be every one beat or every two beats of the pressure pulse wave that is the cyclicity of the data.

The variable data length unit may be a unit based on a boundary between sensing data and time information, for example, in a case where the biological data includes the sensing data by the sensor and the information (e.g., date of measurement and the like) of the time when the sensing data is obtained. For example, when the biological data is pressure pulse wave data, time information may be inserted in some places of the pressure pulse wave data, and an error detection code may be assigned to at least any of before and after the time information. Specifically, the data length unit may have measurement date information, the error detection code, and the pressure pulse wave data sensed are arranged in this order.

The variable data length unit may be a unit based on a stage of measurement of biological data. For example, in a case where the biological data to be measured is pressure pulse wave data, and the measurement is performed in two stages of a pressurization stage and a measurement stage, the variable data length unit may be a data length unit with an error detection code assigned at timing of pressurization start, pressurization end, measurement end, or the like. By assigning the error detection code at the timing of pressurization end, it is possible to obtain pressure pulse wave data during pressurization. By assigning the error detection code at the timing of measurement end, it is possible to obtain pressure pulse wave data after pressurization.

The variable data length unit is a data length different from a transfer unit (e.g., a fixed length unit of transmission of 18 bytes every 32 msec) for transferring the biological data from the main MCU 18 to the communication IC 14. The variable data length unit is a data length different from a transmission unit (unit of fixed length) for wirelessly transmitting the biological data from the communication IC 14 to the external device.

Note that assignment of the error detection code to the biological data may be performed by the communication IC 14. In this case, for example, the main MCU 18 transmits information indicating the variable data length unit to the communication IC 14 based on the content of the biological data. The information indicating the variable data length unit is, for example, a boundary code indicating the boundary between the sensing data and the time information described above. However, the information is not limited to the boundary code as long as the “variable data length unit” can be specified on the communication IC 14 side.

For example, the communication IC 14 assigns an error detection code to the biological data based on the information indicating the variable data length unit transmitted from the main MCU 18. For example, the communication IC 14 may assign an error detection code to the biological data to be written into the nonvolatile memory 14a. Specifically, the communication IC 14 assigns an error detection code before writing the biological data into the nonvolatile memory 14a. The communication IC 14 may assign an error detection code to the biological data read from the nonvolatile memory 14a and transmitted to the external device. Specifically, the communication IC 14 assigns an error detection code when reading the biological data from the nonvolatile memory 14a, and transmits, to the external device, the biological data with the error detection code assigned. Note that the communication IC 14 may separately assign an error detection code in a transmission unit for wirelessly transmitting the biological data from the communication IC 14 to the external device.

By executing, for example, an information output program stored in the nonvolatile memory 18a, the main MCU 18 outputs a biological measurement result based on measured biological data, for example, a blood pressure measurement result based on pressure pulse wave data. The main MCU 18 displays the blood pressure measurement result on a screen of the display unit 11 of the measurement device 1, for example. The main MCU 18 may output the blood pressure measurement result by voice from the measurement device 1 or may wirelessly transmit the blood pressure measurement result to the information terminal 5.

### <Configuration of Information Terminal 5>

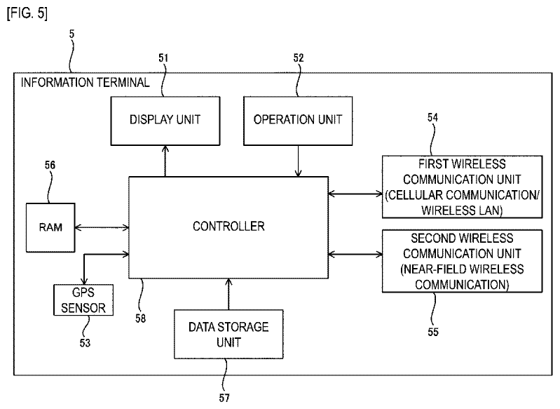

FIG. 5 is a block diagram illustrating the configuration of the information terminal 5. The information terminal 5 includes a display unit 51 that can display various types of information, an operation unit 52 than can be operated by the user, a global positioning system (GPS) sensor 53 for detecting a position, and a first wireless communication unit 54 and a second wireless communication unit 55 that perform communication with an external device. The information terminal 5 includes a RAM 56 that temporarily stores information, a data storage unit 57 that stores information and programs, and a controller 58 that controls the operation of the entire terminal.

The display unit 51 includes a liquid crystal display or an organic electro luminescence (EL) display, for example. The operation unit 52 is a user interface that receives a user operation such as a button or a touch panel. The button includes a button physically provided at the information terminal 5 or a virtual button displayed on the display unit 51. The GPS sensor 53 is a sensor for detecting the current position of the information terminal 5.

The first wireless communication unit 54 is a communication unit that performs cellular communication, for example, a circuit (module) that can perform communication in compliance with a standard such as 4G, 5G, or long term evolution (LTE: registered trademark). The first wireless communication unit 54 is a communication unit that performs wireless LAN communication, for example, a circuit (module) that can perform communication in compliance with a standard such as Wi-Fi (registered trademark). The second wireless communication unit 55 is a communication unit that performs near-field wireless communication, for example, a circuit (module) for performing communication in compliance with the BLE standard.

The second wireless communication unit 55 acquires the biological data of the user measured by the measurement device 1, for example, by performing BLE communication with the communication IC 14 of the measurement device 1. The second wireless communication unit 55 receives the advertise signal transmitted from the communication IC 14 of the measurement device 1 by scanning. The second wireless communication unit 55 recognizes the measurement device 1 from the received advertise signal, and transmits a connection request to the measurement device 1 when communication connection is desired. Note that the measurement device 1 waits for a connection request for a predetermined time after transmitting the advertise signal, stops sending the advertise signal upon receiving the connection request within the predetermined time, and switches to one-to-one connection communication with a connection request partner.

The RAM 56 includes a semiconductor device such as a DRAM or an SRAM, for example, temporarily stores information, and operates as a work area of the controller 58.

The data storage unit 57 is a recording medium that stores a parameter necessary for implementing a predetermined function, a control program, measurement biological data acquired from the measurement device 1, and the like. The data storage unit 57 includes, for example, a hard disk drive (HDD) or a semiconductor storage device (SSD).

The controller 58 implements a predetermined function by executing the control program. Note that in the present embodiment, for example, management application software for an information terminal is installed in advance as a control program in the data storage unit 57, and the controller 58 implements the predetermined function by executing this management application software. For example, when the management application software for the information terminal is started, the controller 58 controls the second wireless communication unit 55 so as to receive the advertise signal by scanning. When the advertise signal from the measurement device 1 is received, the controller 58 transmits a connection request to the measurement device 1 and controls the second wireless communication unit 55 so as to acquire the measurement biological data from the measurement device 1.

### § 3 Operation Example

### <Operation Example of Measurement Device 1>

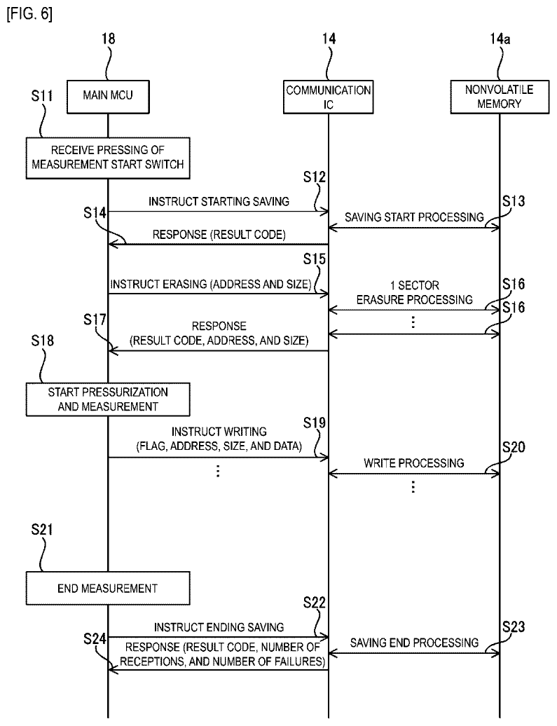

Next, an operation example of the measurement device 1 will be described with reference to FIG. 6. FIG. 6 is a sequence diagram illustrating operations of the main MCU 18, the communication IC 14, and the nonvolatile memory 14a in the measurement device 1. Note that the present example will be described below with the measurement device 1 being the sphygmomanometer 1A, and the bioinformation measured by the sphygmomanometer 1A being pressure pulse wave data.

It is assumed that an upper arm of the user is attached with the cuff 22 of the sphygmomanometer 1A and a measurement start switch is pressed.

First, the main MCU 18 receives pressing of the measurement start switch (step S11). Next, the main MCU 18 transmits, to the communication IC 14, a saving start instruction signal for instructing preparation for writing start (step S12).

Next, in response to the saving start instruction signal received in step S12, the communication IC 14 performs saving start processing for starting writing to the nonvolatile memory 14a (step S13). Next, the communication IC 14 transmits, to the main MCU 18, a response signal notifying that the saving start processing is completed (step S14). The response signal includes a result code indicating that the saving start processing is completed.

Next, upon receiving the response signal in step S14, the main MCU 18 transmits, to the communication IC 14, an erasure instruction signal for erasing the data in the nonvolatile memory 14a (step S15). The erasure instruction signal includes specification of an address of an area to be erased in the nonvolatile memory 14a and the size thereof.

Next, in response to the erasure instruction signal received in step S15, the communication IC 14 performs processing of erasing the data of the instructed area, for example, for each sector (step S16). The communication IC 14 repeats the erasure processing for 1 for each sector in accordance with the size of the instructed erasure area. The erasure processing for each sector is that, for example, when viewed from the communication IC 14 side, the communication IC 14 first transmits an erasure instruction for each sector to the nonvolatile memory 14a, and receives, from the nonvolatile memory 14a, a response indicative of having complied with the erasure instruction (a response indicating that one sector has been erased). Next, the communication IC 14 transmits, to the nonvolatile memory 14a, a read request for reading the erased one sector, receives, from the nonvolatile memory 14a, a response (read data of one sector) in response to the read request, then performs verification, and ends. Next, the communication IC 14 transmits, to the main MCU 18, a response signal notifying that the erasure processing is completed (step S17). The response signal includes a result code indicating that the erasure processing is completed, and an address and a data size of the erased data in the nonvolatile memory 14a.

Next, upon receiving the response signal in step S17, the main MCU 18 pressurizes the cuff 22 and starts measuring the pressure pulse wave data (step S18). The main MCU 18 assigns the pressure pulse wave data obtained by this measurement with an error detection code in a variable data length unit (e.g., for each beat of the pressure pulse wave, and the like) corresponding to the content of the pressure pulse wave data. Next, the main MCU 18 transmits, to the communication IC 14, a write instruction signal for writing the measured pressure pulse wave data into the biological data area of the nonvolatile memory 14a (step S19). The write instruction signal includes a flag indicating transmission (e.g., transmission by a streaming method) in which delivery confirmation as to whether or not the pressure pulse wave data has been delivered is not performed, an address of the nonvolatile memory 14a into which the pressure pulse wave data is written and a data size thereof, and the pressure pulse wave data to be written. The transmission of the pressure pulse wave data in this write instruction is sequentially performed by dividing, at regular time intervals, the pressure pulse wave data measured by the measurement unit 13.

Next, in response to the write instruction signal received in step S19, the communication IC 14 performs write processing of writing the pressure pulse wave data sequentially transmitted from the main MCU 18 into a specified address in the biological data area of the nonvolatile memory 14a for each pressure pulse wave data that is sequentially transmitted (step S20). The write processing for each pressure pulse wave data sequentially transmitted is that for example, when viewed from the communication IC 14 side, the communication IC 14 first transmits, to the nonvolatile memory 14a, a write instruction for each pressure pulse wave data sequentially transmitted from the main MCU 18, and receives, from the nonvolatile memory 14a, a response indicative of having complied with the write instruction (a response indicating that the pressure pulse wave data has been written). Next, the communication IC 14 transmits, to the nonvolatile memory 14a, a read request for reading the written pressure pulse wave data, receives, from the nonvolatile memory 14a, a response (read pressure pulse wave data) in response to the read request, then performs verification, and ends.

Note that in the description of step S18, the main MCU 18 assigns the error detection code to the pressure pulse wave data obtained by the sensing of the measurement unit 13, but no such limitation is intended. For example, assignment of the error detection code may be performed by the communication IC 14. In this case, based on the information indicating the variable data length unit based on the content of the pressure pulse wave data transmitted from the main MCU 18, the communication IC 14 assigns the error detection code, for example, before writing the pressure pulse wave data into the nonvolatile memory 14a in step S20.

Next, upon ending the measurement of the pressure pulse wave data (step S21), the main MCU 18 transmits, to the communication IC 14, a saving end instruction signal instructing processing for ending writing (step S22).

Next, in response to the saving end instruction signal received in step S22, the communication IC 14 performs saving end processing for ending writing to the nonvolatile memory 14a (step S23). Next, the communication IC 14 transmits, to the main MCU 18, a response signal notifying that the saving end processing is completed (step S24). The response signal includes a result code indicating that the saving end processing is completed, the number of receptions of the pressure pulse wave data that the communication IC 14 has received from the main MCU 18, and the number of failures of the pressure pulse wave data having failed to be written to the nonvolatile memory 14a.

The transmission processing for transmitting the pressure pulse wave data written into the nonvolatile memory 14a from the sphygmomanometer 1A to the external information terminal 5 is performed, for example, after the measurement of the pressure pulse wave data described above ends. In this case, the main MCU 18 transmits, to the communication IC 14, a transmission instruction signal for transmitting the pressure pulse wave data. The transmission instruction signal includes specification of an address in the nonvolatile memory 14a of the pressure pulse wave data to be transmitted and the size thereof. Next, the communication IC 14 reads the pressure pulse wave data from the nonvolatile memory 14a in accordance with the transmission instruction signal received from the main MCU 18, and transmits the read pressure pulse wave data to the information terminal 5 by wireless communication. The communication IC 14 transmits, to the information terminal 5, result information (the number of receptions, the number of failures, and the like) regarding writing of the pressure pulse wave data together with the pressure pulse wave data.

### § 4 Error Detection Code of Biological Data

### <Example of Error Detection Code Assigned to Biological Data>

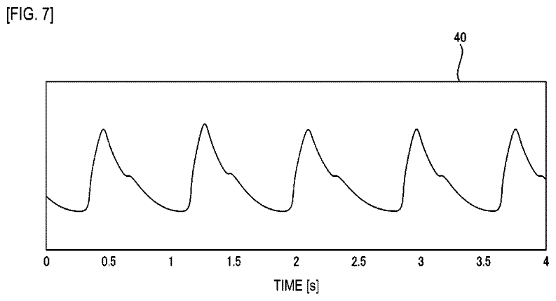

FIG. 7 is a view illustrating an example of biological data to be measured by the measurement device 1. The present example illustrates an example of pressure pulse wave data to be measured by the sphygmomanometer 1A. As illustrated in FIG. 7, pressure pulse wave data 40 is measured as a continuous conduction wave having substantially constant cyclicity. The pressure pulse wave data 40 having been measured is transmitted from the main MCU 18 to the communication IC. The main MCU 18 divides, at regular time intervals, and sequentially transmits, to the communication IC 14, the pressure pulse wave data 40 having been measured. An error detection code in a variable data length unit is assigned to the measured pressure pulse wave data.

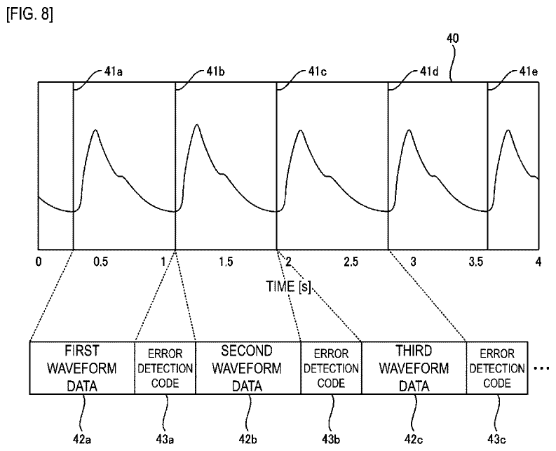

FIG. 8 is a view illustrating an example in which the main MCU 18 assigns an error detection code to the biological data. The main MCU 18 performs pulse wave determination of the pressure pulse wave data 40 having been measured, and detects boundary positions 41a, 41b, 41c, 41d, and 41e for each pressure pulse wave data of one beat as illustrated in FIG. 8 with the data length of the pressure pulse wave of one beat as a data length unit.

For example, the main MCU 18 sets pressure pulse wave data of one beat between the boundary positions 41a and 41b as first waveform data 42a, pressure pulse wave data of one beat between the boundary positions 41b and 41c as second waveform data 42b, and pressure pulse wave data of one beat between the boundary positions 41c and 41d as third waveform data 42c, and the like. Then, the main MCU 18 calculates an error detection code 43a from the first waveform data 42a, and assigns the calculated error detection code 43a to the first waveform data 42a. Similarly, the main MCU 18 assigns an error detection code 43b calculated from the second waveform data 42b to the second waveform data 42b, and assigns an error detection code 43c calculated from the third waveform data 42c to the third waveform data 42c. The main MCU 18 includes, into the write instruction signal, the pressure pulse wave data with the error detection code assigned, that is, the first waveform data 43a with the error detection code 42a assigned, the second waveform data 43b with the error detection code 42b assigned, and the third waveform data 43c with the error detection code 42c assigned, and the like, and transmits the write instruction signal to the communication IC 14.

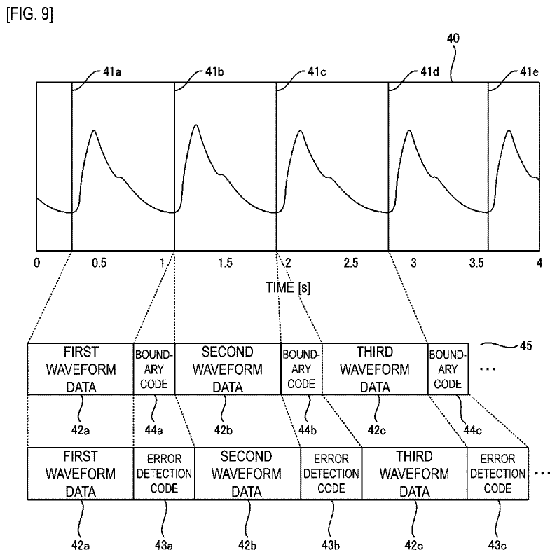

FIG. 9 is a view illustrating an example in which the communication IC 14 assigns an error detection code to the biological data. As illustrated in FIG. 9, a point that with the data length of a pressure pulse wave of one beat being a data length unit, the boundary positions 41a, 41b, 41c, 41d, and 41e are set for each pressure pulse wave data of one beat is similar to the case described with reference to FIG. 8. The point of setting pressure pulse wave data of one beat between the boundary positions 41a and 41b as first waveform data 42a, pressure pulse wave data of one beat between the boundary positions 41b and 41c as second waveform data 42b, and pressure pulse wave data of one beat between the boundary positions 41c and 41d as third waveform data 42c, and the like is also similar to the case described with reference to FIG. 8.

Next, as information indicating the variable data length unit in the pressure pulse wave data, the main MCU 18 transmits information of the boundary positions 41a, 41b, 41c, 41d, and 41e set for each pressure pulse wave data of one beat to the communication IC 14 together with the pressure pulse wave data. For example, the main MCU 18 sets and assigns, between the first waveform data 42a and the second waveform data 42b, information indicating the boundary position 41b between the first waveform data 42a and the second waveform data 42b as a boundary code 44a. Similarly, the main MCU 18 sets and assigns, between the second waveform data 42b and the third waveform data 42c, information indicating the boundary position 41c between the second waveform data 42b and the third waveform data 42c as a boundary code 44b. Similarly, the main MCU 18 sets and assigns, between the third waveform data 42c and the fourth waveform data 42d, information indicating the boundary position 41d between the third waveform data 42c and the fourth waveform data 42d (not illustrated) as a boundary code 44c. The main MCU 18 transmits, to the communication IC 14, the first waveform data 42a, the boundary code 44a, the second waveform data 42b, the boundary code 44b, the third waveform data 42c, the boundary code 44c, and the like (hereinafter, also called “boundary code assigned data 45”).

The communication IC 14 divides, at parts of the boundary codes 44a, 44b, and 44c, the boundary code assigned data 45 received from main MCU 18. The communication IC 14 calculates an error detection code for each divided waveform data. That is, the communication IC 14 calculates error detection code 43a from first waveform data 42a, calculates the error detection code 43b from the second waveform data 42b, and calculates the error detection code 43c from the third waveform data 42c. The communication IC 14 assigns the calculated error detection code 43a to the first waveform data 42a, assigns the error detection code 43b to the second waveform data 42b, and assigns the error detection code 43c to the third waveform data 42c.

When assigning the error detection code 43a, the error detection code 43b, and the error detection code 43c to the first waveform data 42a, the second waveform data 42b, and the third waveform data 42c, respectively, based on the boundary code assigned data 45 received from the main MCU 18, the communication IC 14 assigns the error detection codes, for example, before writing the first waveform data 42a, the second waveform data 42b, and the third waveform data 42c into the nonvolatile memory 14a.

In this case, the main MCU 18 performs address specification (see step S19 of FIG. 6) of the nonvolatile memory 14a in the write instruction signal in consideration of the data size that changes by assigning the error detection code 43a, the error detection code 43b, and the error detection code 43c. However, in a case where the data length of the error detection code is a fixed length, the data lengths of the boundary codes 44a, 44b, and 44c may be set to have the same data lengths as the data lengths of the error detection codes 43a, 43b, and 43c. By setting the same data lengths in this manner, the main MCU 18 can perform address specification of the nonvolatile memory 14a in no consideration of the data size that changes due to the assignment of the error detection code.

In a case of assigning the error detection code 43a, the error detection code 43b, and the error detection code 43c based on the boundary code assigned data 45, the communication IC 14 may assign the error detection code, for example, when reading, from the nonvolatile memory 14a, the first waveform data 42a, the second waveform data 42b, and the third waveform data 42c written in the nonvolatile memory 14a, that is, when transmitting the first waveform data 42a, the second waveform data 42b, and the third waveform data 42c to the information terminal 5 by wireless communication. In this case, the main MCU 18 can perform address specification of the nonvolatile memory 14a in the write instruction signal in no consideration of the data size that changes due to the assignment of the error detection code 43a, the error detection code 43b, and the error detection code 43c.

When transmitting, to the communication IC 14, the first waveform data 42a, the second waveform data 42b, the third waveform data 42c, and the like, the main MCU 18 may transmit information indicating boundary timing (time) to the communication IC 14 separately from the first waveform data 42a, the second waveform data 42b, the third waveform data 42c, and the like, not inserting the boundary codes 44a, 44b, 44c, and the like between the waveform data. The information indicating the boundary timing (time) may be information indicating that, for example, which byte is the boundary between the first waveform data 42a and the second waveform data 42b and which byte is the boundary between the second waveform data 42b and the third waveform data 42c.

As described above, the control unit 30 of the measurement device 1 assigns the biological data obtained by sensing (pressurization and measurement) by the measurement unit 13 with an error detection code in a variable data length unit corresponding to the content of the biological data, and transmits, to the information terminal 5, the biological data with the error detection code assigned. Therefore, for example, in the analysis of the biological data in the information terminal 5 such as a smartphone or a cloud server, even if there is an error in the biological data received from the measurement device 1, the error can be detected in a meaningful data unit corresponding to the content of the biological data. This can improve the efficiency of analysis of the biological data in the information terminal 5. For example, when the biological data obtained by sensing is pressure pulse wave data and an error detection code is assigned for each pressure pulse wave data of one beat, data of one beat of pressure pulse wave data having an error may be discarded, and the pressure pulse wave data of the beats before and after can be used as it is for analysis. If an error detection code is assigned in a data unit (e.g., for each predetermined data size) that is not related to beats, when the data unit includes pressure pulse wave data of a plurality of beats, the pressure pulse wave data of the plurality of beats cannot be used for analysis, and the efficiency of analysis is deteriorated.

The main MCU 18 of the measurement device 1 sequentially transmits, to the communication IC 14, without performing delivery confirmation, and writes, into the nonvolatile memory 14a, the biological data obtained during the sensing by the measurement unit 13, and after the sensing of the biological data ends, receives, from the communication IC 14, the result information related to the writing of the biological data into the nonvolatile memory 14a. The communication IC 14 reads and transmits, to the information terminal 5 by wireless communication, the biological data written in the nonvolatile memory 14a. According to this configuration, by providing the communication IC 14 that performs wireless communication separately from the main MCU 18 that performs measurement based on the biological data, it is possible to distribute, to the communication IC 14, the processing load for transmitting, to the information terminal 5, the biological data such as pressure pulse wave data, and reduce the processing load of the main MCU 18. This can suppress a delay in processing such as measurement by the main MCU 18. The main MCU 18 sequentially transmits, to the communication IC 14, without performing delivery confirmation, the biological data obtained during sensing, and writes the biological data into the nonvolatile memory 14a, whereby the transfer speed of the biological data from the main MCU 18 to the communication IC 14 can be improved. The processing load of the main MCU 18 during sensing can be reduced. By transmitting the result information related to the writing of the biological data to the nonvolatile memory 14a from the communication IC 14 to the main MCU 18 after the end of the sensing, the main MCU 18 can recognize the writing result of the biological data to the nonvolatile memory 14a even in the configuration in which the delivery confirmation is not performed.

In the measurement device 1, the variable data length unit with the error detection code assigned may be, for example, a unit corresponding to the cycle of the biological data to be measured, a unit based on the boundary between the sensing data and the time information, or a unit based on the measurement stage of the biological data obtained by the measurement unit 13. This can further assign the error detection code in a meaningful data unit corresponding to the content of the biological data.

In the measurement device 1, the variable data length unit with the error detection code assigned is, for example, a unit different from the transmission unit of the biological data to the information terminal 5, and is a unit different from the transfer unit of the biological data from the main MCU 18 to the communication IC 14. This can improve the analysis efficiency of the biological data.

In the measurement device 1, the main MCU 18 transmits, to the communication IC 14, information indicating the variable data length unit based on the content of the biological data, and the communication IC 14 assigns the error detection code to the biological data based on the information indicating the variable data length unit. According to this configuration, since the communication IC 14 performs calculation of the error detection code as compared with the case where the main MCU 18 performs determination of the biological data and calculation of the error detection code, it is possible to reduce the load on the main MCU 18. For example, the communication IC 14 assigns an error detection code to the biological data read from the nonvolatile memory 14a and transmitted to the information terminal 5. In this manner, by assigning the error detection code at the time of reading the biological data, the data sent from the main MCU 18 to the communication IC 14 is written into the nonvolatile memory 14a as it is, and therefore the main MCU 18 can perform address specification in the nonvolatile memory 14a in no consideration of the data size that changes due to the assignment of the error detection code.

The main MCU 18 of the measurement device 1 specifies an address of a write destination in the biological data area of the nonvolatile memory 14a and causes the communication IC 14 to write the biological data into the nonvolatile memory 14a, and specifies an address of a read source in the biological data area of the nonvolatile memory 14a and causes the communication IC 14 to read the biological data from the nonvolatile memory 14a and transmit the biological data read from the nonvolatile memory 14a to an external device such as the information terminal 5 by wireless communication. According to this configuration, since the main MCU 18 is configured to specify the address of the nonvolatile memory 14a connected to the communication IC 14 and instruct the communication IC 14 to write, read, and transmit the biological data, flow control and delivery confirmation are unnecessary in the interface between the main MCU 18 and the communication IC 14, and the transfer speed of the biological data to the information terminal 5 can be improved. Since the communication IC 14 may perform writing of information to a specified address in the nonvolatile memory 14a and reading and transmission of information from the specified address in the nonvolatile memory 14a, the communication IC 14 can have a simple configuration. By an instruction to the communication IC 14, the main MCU 18 can flexibly perform writing of the biological data to the nonvolatile memory 14a, reading of the biological data from the nonvolatile memory 14a, and transmission of the read biological data. However, since it is not necessary for the main MCU 18 itself to perform processing with a large load such as write processing, read processing, and transmission processing of biological data and the like, it is possible to reduce the processing load of the main MCU 18 as described above.

The measurement device 1 has a biological data area allocated for storing biological data in the nonvolatile memory 14a. The biological data stored in the biological data area is configured to be managed by the communication IC 14 and not accessible from the main MCU 18. According to this configuration, since information other than the biological data is not written into the biological data area, it is possible to suppress interference between writing of the biological data by an instruction from the main MCU 18 to the communication IC 14 and writing of other information by the communication IC 14. As compared with a configuration in which the main MCU 18 and the communication IC 14 share one nonvolatile memory, access processing can be distributed and sped up.

### § 5 Modified Examples

While an embodiment of the present invention has been described in detail above, the above description is merely illustrative of the present invention in all respects. Various modifications and variations can be made without departing from the scope of the present invention. For example, the following changes are possible. Note that in the following, the same reference signs are used for components that are the same as those of the above-described embodiment, and descriptions thereof are omitted as appropriate. The following modified examples can be combined as appropriate.

In the above embodiment, when the biological data measured by the main MCU 18 is written into the nonvolatile memory 14a and when the biological data measured by the main MCU 18 is read from the nonvolatile memory 14a, the biological data is transmitted to the communication IC 14 by specifying the addresses of the write destination and the read source in the nonvolatile memory 14a, but no such limitation is intended. For example, the main MCU 18 may transmit the biological data to the communication IC 14 without specifying the addresses of the write destination and the read source of the biological data in the nonvolatile memory 14a. In this case, the communication IC 14 performs address management of writing and reading of the biological data in the nonvolatile memory 14a.

In the above embodiment, when the biological data measured by the main MCU 18 is written into the nonvolatile memory 14a, the biological data obtained during measurement by the measurement unit 13 is sequentially transmitted to the communication IC 14 without delivery confirmation and written, but no such limitation is intended. For example, the main MCU 18 may perform delivery confirmation of the biological data every time it sequentially transmits the biological data to the communication IC 14. In that case, since it is possible to obtain, by the delivery confirmation, the result information regarding the writing of the biological data to the nonvolatile memory 14a, it is not necessary to receive the result information from the communication IC 14 after the end of the sensing.

In the above embodiment, the configuration in which the biological data is pulse wave data and the pressure pulse wave data is acquired as pulse wave data has been described, but the measurement device 1 may be configured to measure volume pulse wave data as pulse wave data.

While various embodiments have been described above, needless to say, the present invention is not limited to such examples. It will be apparent to those skilled in the art that various changes and modifications can be made within the scope of the claims, and it should be understood that such changes and modifications also fall within the technical scope of the present invention. Components in the above-described embodiment may be combined as desired without departing from the gist of the invention.

Note that the present application is based on Japanese Patent Application (Japanese Patent Application No. 2023-011008) filed on Jan. 27, 2023, the contents of which are incorporated herein by reference.

### REFERENCE SIGNS LIST

1 Measurement device
1A Sphygmomanometer
5 Information terminal
11, 51 Display unit
12, 52 Operation unit
13 Measurement unit
14 Communication IC (second processor)
14a, 18a Nonvolatile memory
14b Antenna
16, 56 RAM
18 Main MCU (first processor)
21 Main body portion
22 Cuff
23 Air tube
30 Control unit
40 Pressure pulse wave data
41a to 41e Boundary position
42a First waveform data
42b Second waveform data
42c Third waveform data
42d Fourth waveform data
43a to 43c Detection code
44a to 44c Boundary code
45 Boundary code assigned data
53 GPS sensor
54 First wireless communication unit
55 Second wireless communication unit
57 Data storage unit
58 Controller
90 Cloud server
100 Information management system

1. A measurement device comprising:
a control unit that performs measurement based on biological data obtained by a sensor and wireless communication with an information terminal, wherein
the control unit
assigns an error detection code calculated based on the biological data in a variable data length unit corresponding to content of the biological data to the biological data obtained by sensing by the sensor, and
transmits, to the information terminal, the biological data with the error detection code assigned.

2. The measurement device according to claim 1, wherein
the variable data length unit is different from a transmission unit of the biological data to the information terminal.

3. The measurement device according to claim 1, wherein
the biological data has cyclicity, and
the variable data length unit is a unit corresponding to a cycle of the biological data.

4. The measurement device according to claim 1, wherein
the biological data includes sensing data obtained by the sensor and information of time when the sensing data is obtained, and
the variable data length unit is a unit based on a boundary between the sensing data and the information of the time.

5. The measurement device according to claim 1, wherein
the variable data length unit is a unit based on a stage of the measurement.

6. The measurement device according to claim 1, wherein
the control unit includes
a first processor that performs the measurement,
a second processor that performs wireless communication with the information terminal, and
a nonvolatile memory connected to the second processor,
the first processor sequentially transmits, to the second processor, the biological data obtained during sensing by the sensor and writes the biological data into the nonvolatile memory, and
the second processor transmits, to the information terminal, the biological data written in the nonvolatile memory.

7. The measurement device according to claim 6, wherein
the variable data length unit is different from a transfer unit of the biological data from the first processor to the second processor.

8. The measurement device according to claim 6, wherein
the first processor assigns the error detection code to the biological data.

9. The measurement device according to claim 6, wherein
the first processor transmits information indicating the variable data length unit to the second processor based on content of the biological data, and
the second processor assigns the error detection code to the biological data based on information indicating the variable data length unit.

10. The measurement device according to claim 6, wherein
the second processor assigns the error detection code to the biological data to be written into the nonvolatile memory.

11. The measurement device according to claim 6, wherein
the second processor assigns the error detection code to the biological data read from the nonvolatile memory and transmitted to the information terminal.

12. The measurement device according to claim 6, wherein
the first processor
specifies an address of a write destination in the nonvolatile memory and causes the second processor to write the biological data into the nonvolatile memory, and
specifies an address of a read source in the nonvolatile memory and causes the second processor to read the biological data from the nonvolatile memory and transmit the biological data to the information terminal.

13. The measurement device according to claim 12, wherein
the nonvolatile memory has an area allocated for the biological data, and
the address of the write destination and the address of the read source are addresses in the area.

14. The measurement device according to claim 6, wherein
the nonvolatile memory is inaccessible from the first processor.

15. The measurement device according to claim 1, wherein
the biological data is pulse wave data.

16. The measurement device according to claim 15, wherein
the control unit outputs a blood pressure measurement result based on the pulse wave data.

17. A control method of a measurement device including a control unit that performs measurement based on biological data obtained by a sensor and wireless communication with an information terminal,
wherein
the control unit
assigns an error detection code calculated based on the biological data in a variable data length unit corresponding to content of the biological data to the biological data obtained by sensing by the sensor, and
transmits, to the information terminal, the biological data with the error detection code assigned.

18. A control recording medium of a measurement device including a control unit that performs measurement based on biological data obtained by a sensor and wireless communication with an information terminal, the control recording medium for causing the control unit to execute processing of
assigning an error detection code calculated based on the biological data in a variable data length unit corresponding to content of the biological data to the biological data obtained by sensing by the sensor, and
transmitting, to the information terminal, the biological data with the error detection code assigned.
